(19) United States
(12) Patent Application Publication
(10) Pub. No.: US 2019/0175558 A1
(43) Pub. Date: Jun. 13, 2019

(54) TOPICAL PHARMACEUTICAL COMPOSITIONS FOR TREATMENT OF TISSUE DAMAGE

(71) Applicant: S.L.A. PHARMA AG, Liestal (CH)

(72) Inventors: David Nigel ARMSTRONG, Lawrenceveille, GA (US); Justin SLAGEL, Watford (GB)

(21) Appl. No.: 16/211,321

(22) Filed: Dec. 6, 2018

Related U.S. Application Data
(60) Provisional application No. 62/595,632, filed on Dec. 7, 2017.

(51) Int. Cl. A61K 31/4164; A61K 47/06; A61K 9/06; A61K 45/06; A61P 17/02

(52) U.S. Cl. CPC A61K 31/4164; A61K 47/06; A61P 17/02; A61K 45/06; A61K 9/06

(57) ABSTRACT

A topical composition comprising about 5 wt % to about 25 wt % of metronidazole or a pharmacologically acceptable salt thereof in a carrier. The composition may be used in the treatment of tissue damage from surgery, infections and other damaging diseases. One advantage of the composition is that topical administration of metronidazole results in a primarily local effect, and thus, side effects observed from systemic administration are avoided.

### CROSS REFERENCE TO RELATED APPLICATION

This application claims priority to co-pending U.S. Provisional Patent Application Ser. No. 62/595,632 filed on Dec. 7, 2017, the contents of which are incorporated by reference herein for all purposes.

### BACKGROUND OF THE INVENTION

### Technical Field

The present invention relates to a pharmaceutical composition, in particular a topical composition comprising metronidazole or a pharmacologically acceptable salt thereof, for the treatment of multiple different types of tissue damage accessible for use of a topical composition.

### Related Art

Metronidazole (or “Flagyl”) is a synthetic antibacterial and antiprotozoan antibiotic having the formula 2-methyl-5-nitroimidazole-1-ethanol. Metronidazole possesses not only anti-bacterial properties, but also anti-inflammatory properties, which are less well understood. The medication is used for its anti-inflammatory properties in the treatment of several skin or tissue damage due surgery or skin diseases.

However, in its oral and intravenous forms, metronidazole is frequently associated with a number of serious side effects. These negative side effects include GI manifestations, such as nausea, vomiting, a metallic taste in the mouth, or inflammation of the oral cavity. Serious neurological side effects can occur which usually manifest as numbness or tingling of the extremities. These neurological side effects can be debilitating, are often irreversible, and necessitate stopping the Metronidazole. Serious hematological, cardiovascular, or renal complications are also common and can be life-threatening. In addition, the overgrowth of opportunistic organisms such as Candida can result from oral or intravenous metronidazole treatment. In addition, oral metronidazole can interact in an adverse manner with other medications, such as oral anticoagulants (e.g. coumadin), which can cause potentially fatal bleeding.

Topical metronidazole has previously been used for a number of skin conditions (e.g. rosacea) or as a topical vaginal preparation in the treatment of vaginal infections (e.g. trichomonas). However, these preparations are contained in a medium containing alcohol, which would result in stinging and burning when used on damaged skin or exposed tissue in a wound. Thus, it would be advantageous to provide a topical composition that overcomes the shortcomings of the prior art.

### SUMMARY OF THE INVENTION

It is, therefore, an object of the present invention to topically apply metronidazole to treat inflammatory skin conditions; skin diseases, tissue damage, including thermal, chemical, radiation burns; infections, and avoid the unwanted side effects of previous compositions and methods of administration.

Metronidazole, 2-methyl-5-nitroimidazole-1-ethanol, is a nitroimidazole derivative with activity against anaerobic protozoa, aerobic and microaerophilic bacteria, having the following structure.

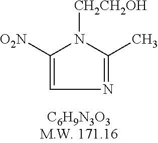

In one aspect, the present invention provides a method for treating tissue damage comprising topically administering to a damaged area of a subject in need thereof, a therapeutically effective amount of a composition comprising from about 5% to 25% of metronidazole or pharmaceutically acceptable salt thereof; and a pharmaceutically acceptable excipient or carrier.

In another aspect, the present invention relates to methods of controlling or alleviating pain by reducing the severity of inflammation and edema associated with damaged tissue accessible for topical application wherein the method comprises: administering to a subject in need thereof a therapeutically effective amount of a pharmaceutical composition comprising metronidazole; and a pharmaceutically acceptable excipient, wherein said pharmaceutical composition inhibits one or more components of the inflammatory pathway.

In a still further aspect, the present invention relates to method for treating numerous tissue damage or diseases, including pilonidal cyst and sinus; anorectal Crohns disease including ulcers, fissures, fistulas, skin tags, etc.; post anorectal surgery including hemorrhoidectomy, fistulotomy or incision drainage abscess; thrombosed external hemorrhoid; perineal incision after abdominoperineal resection; pyoderma gangrenosum; erythema nodosum; hidradenitis; non healing surgical incisions; chemical burns, electrical burns or thermal burns; Bartholin's cysts, sinus or abscess; pruritus ani/perianal folliculitis; decubitus ulcers such as bed sores or pressure sores; perianal/sacral ulcers due to HIV, radiation ulcers, ischemic ulcers, and/or malnutrition ulcers; animal or human bites, skin abrasions, lacerations, cellulitis, superficial thrombophlebitis, and loss of skin integrity.

In yet another aspect, the present invention provides a topical composition comprising metronidazole or a pharmacologically acceptable salt thereof at a concentration of about 5 wt % to about 25 wt % in a pharmacologically acceptable non-aqueous carrier. The concentration of metronidazole is preferably about 10 wt %. The concentrations are based on the total weight of the composition. In some embodiments, the metronidazole is the sole active agent.

The topical composition is preferably in a form suitable for direct application to the damaged tissues. Suitable forms include an enema, suppository, ointment, lotion, gel, foam or cream. Examples of suitable carriers for enema compositions include but are not limited to sodium chloride and 1% methylcellulose; propylene glycol and 2% methylcellulose gel; glycerin, 2% methylcellulose gel (retention enema); base C, glycerin, green soap; silica gel (micronized), 2% methylcellulose gel; 2% methylcellulose gel and purified water, sodium chloride, purified water, sodium hydroxide; xanthan gum, purified water, 2% methylcellulose gel; carbopol, xanthan gum; methocel 1%, polysorbate.

More preferably, the carrier is an organic carrier and, typically, comprises at least one hydrocarbon compound. Preferably, the carrier comprises a mixture of at least two semi-solid saturated hydrocarbon compounds. An example of a suitable carrier is white petrolatum (USP), also known as white soft paraffin (BP). Other suitable carriers include zinc oxide, Vaseline™, Aquaphor (a combination of mineral oil, petrolatum ceresin and lanolin), lanolin or a petroleum-based carrier.

The composition may consist essentially of metronidazole and the carrier. However, a therapeutic amount of at least one other therapeutic agent may be added to the composition to add to its effectiveness. Additional therapeutic agents that may be added include calcium channel blockers, e.g. nifedipine and diltiazem; steroids, e.g. hydrocortisone or a pharmacologically acceptable derivative thereof; analgesic agents, preferably from the amide or ester class such as pramoxine or benzocaine; antimicrobial agents (antibacterial or antiviral), e.g. ciprocfloxacin, amoxicillin-clavulonic acid, erythromycin, tetracycline, clindamycin or doxycycline; substances that either promote skin integrity or inhibits skin breakdown, e.g. vitamin E, aloe, zinc oxide or other barrier cream; anti-inflammatory agents, e.g. a non-steroidal anti-inflammatory agent selected from aminosalicylic acid, ibuprofen, sulindac, piroxicam or diflunisal.

The topical composition is preferably in a form suitable for direct application to the damaged area of the tissue. Suitable forms include ointment, lotion, gel, foam or cream to be used for treatment of tissue damage in warm-blooded animals, such as mammals and especially humans. In particular, the present invention is concerned with inflammation-associated tissue damage and is particularly directed to prophylactic and therapeutic methods for treating localized and systemic wounds or inflammation associated tissue damage described herein.

In yet another aspect, the present invention provides a topical composition comprising metronidazole or a pharmacologically acceptable salt thereof at a concentration of about 5 wt % to about 25 wt %, more preferably about 10 wt %, in a pharmacologically acceptable non-aqueous carrier for use in the topical treatment of the damaged surface skin and tissues. Notably, the metronidazole composition of the present invention relieves pain, reduces inflammation and edema, promotes wound healing and reverses tissue induration and granulation.

In another aspect, the present invention provides for the use of metronidazole or a pharmacologically acceptable salt thereof in the manufacture of a topical medicament to promote healing and relieve pain caused by damaging conditions of skin surfaces and tissues therebeneath.

In yet another aspect, the present invention provides for a method for preventing or ameliorating the adverse effects associated tissue damage due to multiple causes by administering to a subject in need thereof a therapeutically effective amount of a pharmaceutical composition comprising metronidazole or a functional derivative thereof; and a pharmaceutically acceptable excipient, wherein said pharmaceutical composition promotes rapid regeneration of damaged tissues while retaining the original composition of the tissue and minimizing complications and scarring associated with such tissue damage.

In a further aspect, the present invention provides the use of metronidazole or a pharmacologically acceptable derivative thereof in the manufacture of a topical medicament to treat ulcers or skin defects. The ulcers may be infective or inflammatory ulcers and may be induced by HIV or radiation. Further, the ulcers may include pressure sores, varicose ulcers, ischemic ulcers and diabetic ulcers.

The invention also encompasses methods of treatment of the above-mentioned conditions and indications using the topical composition containing metronidazole of present invention. The dose of metronidazole for each application is preferably between from about 125 mg to about 1250 mg, more preferably between from about 125 mg to about 375 mg and most preferably about 250 mg. The most preferred dose is based on a single application of 2.5 cm³ of a 10 wt % metronidazole ointment. The composition is usually applied between from 2 to 4 times daily and preferably 3 times daily.

In another aspect, the present invention provides for the use of metronidazole or a pharmacologically acceptable salt thereof at a concentration of about 5 wt % to about 25 wt %, more preferably about 10 wt %, in a pharmacologically acceptable carrier in the treatment of tissue damage, wherein the tissue damage is a result of pilonidal cyst and sinus; anorectal Crohns disease including ulcers, fissures, fistulas, skin tags, etc.; post anorectal surgery including hemorrhoidectomy, fistulotomy or incision drainage abscess; thrombosed external hemorrhoid; perineal incision after abdominoperineal resection; pyoderma gangrenosum; erythema nodosum; hidradenitis; non healing surgical incisions; chemical burns, electrical burns or thermal burns; Bartholin's cysts, sinus or abscess; pruritus ani/perianal folliculitis; decubitus ulcers such as bed sores or pressure sores; perianal/sacral ulcers including HIV ulcers, radiation ulcers, ischemic ulcers, and/or malnutrition ulcers; animal or human bites, skin abrasions, lacerations, cellulitis, superficial thrombophlebitis, and loss of skin integrity.

In yet another aspect, the present invention relates to kits for the treatment of damaged tissue, wherein the kit includes packaging that contains a composition formulated for topical application and comprising at least an effective amount of metronidazole or salt thereof in a pharmaceutically acceptable carrier.

The composition of the present invention can be used topically by applying over an area to be treated. A typical method of use is to apply or rub the composition over the entire area, until the composition disappears. Several methods are available for the dispensing of the composition on the tissue damage including by physical means including applicator pads, swabs, or other devices intended to apply the composition in a thin film such as roller bottles, felt tip or sponge tip applicators.

Roll on bottles are especially advantageous. The roll-on bottle greatly simplifies the dispensing of the composition on the tissue damage. No hand or finger rubbing is required. The movement of the roller ball on the surface massages the composition over the damaged tissue area. Further, the roller-ball provides a more precise control where the composition is to be applied, to avoid contact with eyes, contact lenses, tender skin, clothing, etc.

These and other advantages and features of the present invention will be described more fully in a detailed description of the preferred embodiments which follows.

### DETAILED DESCRIPTION OF THE INVENTION

Throughout the instant specification and claims, the following definitions and general statements are applicable.

As used herein, whether in a transitional phrase or in the body of a claim, the terms “comprise(s)” and “comprising” are to be interpreted as having an open-ended meaning. That is, the terms are to be interpreted synonymously with the phrases “having at least” or “including at least.” When used in the context of a process, the term “comprising” means that the process includes at least the recited steps, but may include additional steps. When used in the context of a composition, the term “comprising” means that the composition includes at least the recited features or components, but may also include additional features or components.

The terms “consists essentially of” or “consisting essentially of” have a partially closed meaning, that is, they do not permit inclusion of steps or features or components which would substantially change the essential characteristics of a process or composition; for example, steps or features or components which would significantly interfere with the desired properties of the compositions described herein, i.e., the process or composition is limited to the specified steps or materials and those which do not materially affect the basic and novel characteristics of the invention.

The terms “consists of” and “consists” are closed terminology and allow only for the inclusion of the recited steps or features or components.

As used herein, the singular forms “a,” “an” and “the” specifically also encompass the plural forms of the terms to which they refer, unless the content clearly dictates otherwise.

The term “about” is used herein to mean approximately, in the region of, roughly, or around. When the term “about” is used in conjunction with a numerical range, it modifies that range by extending the boundaries above and below the numerical values set forth. In general, the term “about” or “approximately” is used herein to modify a numerical value above and below the stated value by a variance of 10%.

As used herein, “treating” means reducing, hindering or inhibiting the development of, controlling, alleviating and/or reversing the symptoms in the individual to which a combination or composition of the invention has been administered, as compared to the symptoms of an individual not being treated according to the invention. A practitioner will appreciate that the combinations, compositions, dosage forms and methods described herein are to be used in concomitance with continuous clinical evaluations by a skilled practitioner to determine subsequent therapy.

Without wishing to be bound by any particular theory, it is believed that the use of metronidazole by direct application to the diseased or otherwise affected is primarily a local effect. Minimal systemic absorption is observed and therefore systemic side effects are effectively reduced or eliminated. As such, the dose of metronidazole can be altered for specific tissue and applied directly to the diseased or otherwise effected area thereby increasing the efficacy of the medication.

The topical compositions of the present invention are generally cream or viscous suspensions having at least one active ingredient and at least one additional component including preservatives, chelating agents, surfactants, thickeners, thickeners-solubilizers, buffers, co-solvents, or lubricants.

According to one embodiment of the present invention, the pharmaceutical composition takes the form of a cream, ointment or foam suspension that is applied topically to a damaged skin surface. The compositions comprise metronidazole dissolved or dispersed in a suitable flowable carrier. The composition can be thickened with one or more thickeners, can contain a buffer, and can also comprise an effective amount of a lubricant such as a natural or synthetic fat or oil, e.g. a tris-fatty acid glycerate or lecithin. Non-toxic non-ionic surfactants can also be included as wetting agents and dispersants. Preferably, the composition does not include an alcohol because of the painful effect caused by alcohol on damaged skin. Further, the pH of the composition is preferably 5.5 to 8.5, and more preferably a pH of about 7.

The composition according to the present invention may also comprise additional pharmaceutically acceptable compounds and/or compositions. It is thus to be understood that all the additional compounds and/or compositions mentioned below have to be physiologically acceptable.

The composition according to the present invention may be topically applied as such within a suitable carrier, solvent, dissolvent, extract, solutions e.g. oily, suspension; microemulsion, vesicles, etc. Where employed, the carrier is inert in the sense of not bringing about a deactivation or oxidation of the metronidazole, and in the sense of not bringing about any adverse effect on the skin areas to which it is applied.

In one aspect of the invention, the metronidazole is applied in admixture with a dermatologically acceptable carrier (e.g., as a lotion, cream, ointment, soap or the like) so as to facilitate topical application and, in some cases, provide additional therapeutic effects as might be brought about, e.g., by moisturizing of the affected and/or damaged skin. The metronidazole carrier for dermatological compositions preferably comprises a carrier which will form a film or layer on the skin to which it is applied so as to localize the application and provide some resistance to washing off by immersion in water or by perspiration.

Many preparations are known in the art, and include lotions containing oils and emollients such as hydrocarbon oils and waxes, silicone oils, vegetable, animal or marine fats or oils, glyceride derivatives, fatty acids or fatty acid esters, lanolin and derivatives, wax esters, sterols, phospholipids and the like, and generally also emulsifiers (nonionic, cationic or anionic), although some of the emollients inherently possess emulsifying properties. These same general ingredients can be formulated into a cream rather than a lotion, or into gels, by utilization of different proportions of the ingredients and/or by inclusion of thickening agents such as gums or other forms of hydrophilic colloids.

Various types of other ingredients may be present in the metronidazole compositions of the present invention. For example, sunscreens may be included such as those materials commonly employed to block ultraviolet light. Illustrative compounds are the derivatives of PABA, cinnamate and salicylate. The exact amount of sunscreen employed in the compositions can vary depending upon the degree of protection desired from the sun's UV radiation.

The compositions for use in the methods of the present invention may include components suitable as carriers, such as starches, emollients, sugars, microcrystalline cellulose, diluents, granulating agents, lubricants, surfactants including amphoteric, binders, disintegrating agents, and the like, with the topical preparations being preferred.

Emollients are often incorporated into the therapeutic compositions of the present invention. Levels of such emollients may range from about 0.5% to about 60%, preferably between about 5% and 30% by weight of the total composition. Emollients may be classified under such general chemical categories as esters, fatty acids and hydrocarbons. Esters may be mono- or di-esters. Acceptable examples of fatty di-esters include dibutyl adipate, diethyl sebacate, diisopropyl dimerate, and dioctyl succinate. Acceptable branched chain fatty esters include 2-ethyl-hexyl myristate, isopropyl stearate and isostearyl palmitate. Acceptable tribasic acid esters include triisopropyl trilinoleate and trilauryl citrate. Acceptable straight chain fatty esters include lauryl palmitate, myristyl lactate, oleyl eurcate and stearyl oleate.

Suitable fatty acids include those compounds having from 10 to 20 carbon atoms. Especially preferred are compounds such as cetyl, arachidyl, behenyl, cetearyl, myristyl, palmitic and stearyl acids.

Exemplary hydrocarbons which may serve as emollients are those having hydrocarbon chains anywhere from 12 to 30 carbon atoms. Specific examples include mineral oil, petroleum jelly, paraffin oil, squalene and isoparaffins.

Another category of functional ingredients within the therapeutic compositions of the present invention are thickeners. A thickener will usually be present in amounts anywhere from 0.1% to 20% by weight, preferably from about 0.5% to 10% by weight of the composition. Exemplary thickeners are cross-linked polyacrylate materials. Gums may be employed such as xanthan, carrageenan, gelatin, karaya, pectin and locust beans gum. Under certain circumstances the thickening function may be accomplished by a material also serving as a silicone or emollient. For instance, silicone gums having a viscosity in excess of 10 mPas and esters such as glycerol stearate have dual functionality.

Still further, the therapeutic compositions of the present invention may include preservatives, moisturizers, surfactants, antimicrobials, etc. Preservatives may include tetrasodium ethylene-diamine tetraacetic acid (EDTA), methylparaben, benzophenone-4, methylchloroisothiazolinone, sodium benzoatemethylisothiazolinone, and the like, and mixtures thereof. Preservatives, when used, are typically present in an amount from about 0.01% to 10% weight, preferably about 0.05% to 4% weight, and more preferably, from about 0.1% to 2% weight.

Preferred moisturizers may include wheat protein (e.g., laurdimonium hydroxypropyl hydrolyzed wheat protein), hair keratin amino acids, sodium peroxylinecarbolic acid, panthenol, tocopherol (Vitamin E), dimethicone, arachidylglucoside and the like, and mixtures thereof. Moisturizers, when used, are typically present in an amount from about 0.01% to 10% weight, preferably about 0.05% to 1.5% weight, more preferably, from about 0.1% to 1% weight of the composition.

Preferred surfactants, including both the foaming and non-foaming type, include sodium laureth sulfate, sodium laureth-13 carboxylate, disodium laureth sulfosuccinate, disodium cocoamphodiacetate, glycol stearate, PEG-150 distearate and the like, and mixtures thereof. More preferably, at least one amphoteric surfactant is included in the composition, selected from the group consisting of lauroamphocarboxypropionate, lauroamphopropionate, lauroamphoglycinate, lauroamphocarboxyglycinate, lauroamphopropylsulfonate, lauroamphocarboxypropionic acid, myristoamphocarboxy-propionate, myristoamphopropionate, myristoamphoglycinate, myristoamphocarboxyglycinate, myristoamphopropylsulfonate, myristoamphocarboxypropionic acid, cocoamphocarboxypropionate, cocoamphopropionate, cocoamphoglycinate, cocoamphocarboxyglycinate, cocoamphopropylsulfonate, cocoamphocarboxypropionic acid and mixtures thereof. The surfactant component may be present in an amount from about 0.1% to about 20% weight of the composition.

The compounds of the present invention include pharmaceutically acceptable salts that can be prepared by those of skill in the art. As used herein, by “pharmaceutically acceptable salt” it is meant those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art, such as hydrochloride, hydrobromide, mesylate, acetate, trifluoroacetate, propionate, fumarate, tartrate, citrate, phosphate, succinate, bisulfate, etc.

The topical skin treatment composition of the invention can be formulated as a lotion having a viscosity of from 4,000 to 10,000 mPas, a fluid cream having a viscosity of from 10,000 to 20,000 mPas or a cream or a gel having a viscosity of from 20,000 to 100,000 mPas or above.

The metronidazole composition can be packaged in a suitable container to suit its viscosity and intended use by the consumer. For example, a lotion or fluid cream can be packaged in a bottle, a propellant-driven aerosol device or a container fitted with a pump suitable for finger operation. When the composition is a cream, it can simply be stored in a non-deformable bottle or squeeze container, such as a tube or a lidded jar.

Generally, in the practice of methods of the invention, the composition is topically applied to the damaged tissue in a predetermined or as-needed regimen either at intervals by application of a lotion or the like, it generally being the case that gradual improvement is noted with each successive application.

Because of its ease of administration, a cream, lotion, gel or ointment represents the most advantageous topical dosage unit form, and such forms may be prepared as rinse-off or leave-on products, as well as two stage treatment products for use with other skin cleansing or managing compositions. Each of these forms is well understood by those of ordinary skill in the art, such that dosages may be easily prepared to incorporate the pharmaceutical composition of the invention.

In general, the compositions of the present invention are intended to be applied topically and directly to the damaged tissue, as described above. When the wound is deep and/or severe, it is preferred that the composition is in the form of an ointment, salve or cream which is spread directly onto the wound and then covered with a standard sterile dressing pad or other appropriate dressing material. Alternatively, the ointment, cream or salve of the present composition is applied directly onto the dressing pad or other appropriate dressing material. The pad or dressing material is then placed over the wound or burn with the medicine-side down. This latter approach works better when applying dressing to severe burns and shallow wounds.

Thus, the pharmaceutical composition of the present invention is applied to a wound so as to cover the injured surface completely. Dressing-change schedules are of course dictated by the condition of the wound. Dressings are advantageously changed three to four times a day. Repeated daily dressing changes are continued until the wound or burn is healed. Healing time varies, depending upon the type and depth of the wound or the severity of the burn.

The present pharmaceutical composition is effective in the treatment of a large variety of tissue damage including the following:
A pilonidal cyst which is an accumulation of hair beneath the skin of an individual in the pre-sacral region;
Anorectal Crohns disease consists of a number of diverse pathologic features including ulcers, fissures, fistulas, skin tags, etc. Oral metronidazole is the first-line treatment for anorectal Crohns disease, but the tablets are difficult to ingest and the medication often produces systemic side effects such as peripheral neuropathy;
Post anorectal surgery including hemorrhoidectomy, fistulotomy, incision drainage abscess, etc.;
A thrombosed external hemorrhoid is a perianal blood clot which usually develops after straining or heavy lifting;
Perineal incision wherein abdoinoperineal resection is performed for a distal rectal cancer and results in a permanent end colostomy, and removal of the entire rectum and sphincter complex. The perineal incision is sutured close but frequently fails to heal or heals very slowly;
Pyoderma gangrenosum is a necrotizing, suppurating painful skin disease found in patients with inflammatory bowel disease, often around the patients stoma;
Erythema nodosum is a discoid erythematous lesion typically found in the pre tibial region and found in the setting of inflammatory bowel disease;
Chemical burns, electrical burns or thermal burns:
Hidradenitis is a chronic debilitating skin disorder of the perianal/inguinal/axillary area. The condition is caused by inflammation of the apocrine glands of the skin. Traditional treatment involves extensive and painful surgical resection of the diseased skin, with subsequent prolonged packing of the wound or split skin grafts;
Non healing surgical incisions are frequent especially after colectomy or bowel resection;
Bartholin's cyst or abscess is an infection of the perivulvar Bartholin gland and presents as a painful vulvar swelling and redness;
Pruritus ani/perianal folliculitis, wherein pruritus is an intractable itching in the perianal region, thought to result from bacterial colonization of the perianal hair follicles, or micro-infection of the hair, sweat or sebum glands in the perianal region. Treatment is usually with zinc oxide, calmoseptine or steroid creams;
Decubitus ulcers result from prolonged pressure upon the presacral region including prolonged immobility or bedrest, such as in ICUs, nursing homes, stroke units, etc; and
Cutaneous ulcers may occur at several locations in the perianal area, and other areas in the body as a result of HIV, radiation, ischemic, venous and malnutrition.

The present medicinal composition can of course also be used to treat other mammals, such as veterinary animals including, without limitation, dogs, cats, other household pets, horses, farm animals, and the like. The magnitude of a prophylactic or therapeutic dose of the pharmaceutical composition of the invention in the acute or chronic management of pathology and pain associated with above-mentioned indications will vary with the severity of the condition to be treated and the route of administration. The dose, and perhaps the dose frequency, will also vary according to the age, body weight, and response of the individual.

Actual dosage levels of active ingredients in the pharmaceutical compositions of this invention may be varied so as to obtain an amount of the active compound(s) that is effective to achieve the desired therapeutic response for a particular patient, compositions, and mode of administration. It is within the skill of the art to start doses of the pharmaceutical compound at levels lower than required to achieve the desired therapeutic effect and to gradually increase the dosage until the desired effect is achieved.

### Examples

Method of Production of the Composition

100 g of metronidazole powder (USP) was mixed with 900 g of white petrolatum (USP) and the mixture passed through a mixer known as an “ointment mill” to produce a 10 wt % metronidazole composition having a “fluffy” texture.

Pilonidal Cyst/Sinus/Abscess

A pilonidal cyst is an accumulation of hair beneath the skin of an individual in the pre-sacral region (natal cleft). The name derives from pilo (hair) and nidal (nest). The inventors have shown the use of 10% topical metronidazole resulted in complete healing and epithelialization in 20 out of 21 (95%) of patients by 6 weeks, with 3 (14%) late recurrences 4-24 months after treatment.

Thrombosed External Hemorrhoid

A thrombosed external hemorrhoid is a perianal blood clot which usually develops after straining or heavy lifting. Typically these are excised in the office under local anesthesia, however topical 10% metronidazole results in resolution of the hematoma typically in 4-5 days. A 34 yr old female presented the day before Thanksgiving with a large thrombosed external hemorrhoid. Not wishing to undergo surgery, she was treated with topical metronidazole. She returned 5 days later and the thrombus had resolved dramatically.

Perineal Incision after Abdominoperineal Resection

Abdoinoperineal resection is performed for a distal rectal cancer and results in a permanent end colostomy and removal of the entire rectum and sphincter complex. The perineal incision is sutured close but frequently fails to heal or heals very slowly. Application of 10% metronidazole to the perineal incision in a 54 yr old male resulted in rapid healing within 2 weeks.

Pyoderma Gangrenosum

Pyoderma gangrenosum is a necrotizing, suppurating painful skin disease found in patients with inflammatory bowel disease, often around the patient's stoma. A 30 year old with an end ileostomy after proctocolectomy for Crohns disease developed pyoderma around the stoma. He was treated with topical 10% metronidazole and the lesion improved dramatically after 2 weeks therapy

Erythema Nodosum

Erythema nodosum is a discoid erythematous lesion typically found in the pretibial region, in the setting of inflammatory bowel disease. Treatment of a 23 yr old female with Crohns disease resulted in resolution of the lesions with 4 weeks of treatment.

Hidradenitis

Hidradenitis is a chronic debilitating skin disorder of the perianal/inguinal/axillary area. The condition is caused by inflammation of the apocrine glands of the skin. Traditional treatment involves extensive and painful surgical resection of the diseased skin, with subsequent prolonged packing of the wound or split skin grafts. A 30 yr old patient presented with hidradenitis of the perianal and inguinal region. He was treated with 10% metronidazole for 6 weeks and showed near complete resolution of the diseased skin.

Non Healing Surgical Incisions

Slow or non-healing skin incisions are frequent especially after colectomy or bowel resection. A 54 yr old male presented with induration redness and edema of his midline incision 2 weeks after sigmoid colectomy. He was treated with topical 10% metronidazole and showed complete healing after 10 days treatment.

Cutaneous Burns (First, Second, Third Degree)

Cutaneous burns (thermal, chemical or radiation) are typically treated with an inactive compound of silver sulfadiazine, which acts as an antibacterial barrier cream against evaporative fluid loss. A 23 year old male was treated with 10% metronidazole after experiencing first degree thermal burns over 5% of his body surface. The medication resulted in a rapid decrease in redness, swelling and pain in the area, compared to the standard treatment.

Bartholin's Cyst/Sinus/Abscess

Bartholin's cyst or abscess is an infection of the perivulvar Bartholin gland and presents as a painful vulvar swelling and redness. Typical treatment is incision and drainage, followed by a course of systemic antibiotics. A 34 year old female presented to the ER with an infected, indurated Barholin gland and was treated with topical 10% metronidazole. She experienced rapid resolution of her pain and swelling within 10 days of initiating treatment

Pruritus Ani/Perianal Folliculitis

Pruritus is an intractable itching in the perianal region, thought to result from bacterial colonization of the perianal hair follicles, or micro-infection of the hair, sweat or sebum glands in the perianal region. Treatment is usually with zinc oxide, calmoseptine or steroid creams. A 34 year old male presented with intractable pruritus refractory to multiple creams. He was treated with topical 10% metronidazole and experienced a rapid and complete resolution of his symptoms.

Decubitus Ulcers (Bed Sores; Pressure Sores)

Decubitus ulcers result from prolonged pressure upon the presacral region due to prolonged immobility or bedrest, such as in ICUs, nursing homes, stroke units etc. These ulcers are a result of pressure necrosis and manifest as loss of tissue in the cutaneous/subcutaneous or deep fascial planes. The microbiology in these ulcers are gram negative anaerobic flora, and topical 10% metronidazole results in rapid healing of these ulcers. An 86-year-old patient suffered a deep capsule stroke resulting in a deep hemiplegic paralysis and inability to ambulate. A superficial decubitus ulcer resulted, which was treated with 10% topical metronidazole, as well as regular turning by the nursing staff. Four weeks later the ulcer had re-epithelialized and tissue restoration occurred.

Perianal/Sacral Ulcers (HIV Ulcers, Radiation Ulcers, Ischemic (Arterial) or Venous Ulcers, Malnutrition Ulcers)

Cutaneous ulcers may occur at several locations in the perianal area, and other areas in the body as a result of HIV, Radiation ulcers, ischemic ulcers, venous ulcers and malnutrition ulcers. These are a result of immunosuppression, malnutrition or tissue damage from extrinsic sources such as radiation, diminished arterial perfusion or venous congestion. A 65 year old female experienced a saphenous ulcer on the medial aspect of the tibial regions as a result of saphenous venous incompetence. Topical 10% metronidazole was applied, as well as compression hose, which resulted in healing of the ulcer in 4 weeks

That which is claimed is:

1. A method of relieving pain and/or promoting wound healing in a patient due to tissue damage, the method comprising:
applying to the tissue damage area a topical composition, wherein the topical composition comprises metronidazole in a therapeutically effective concentration of from about 5 wt % to 25 wt % in a pharmacologically acceptable carrier.

2. The method of claim 1, wherein the pharmacologically acceptable carrier is non-aqueous.

3. The method of claim 1, wherein the tissue damage is a result of pilonidal cyst and sinus; anorectal Crohns disease including ulcers, fissures, fistulas, skin tags, etc.; post anorectal surgery including hemorrhoidectomy, fistulotomy or incision drainage abscess; thrombosed external hemorrhoid; perineal incision after abdominoperineal resection; pyoderma gangrenosum; erythema nodosum; hidradenitis; non healing surgical incisions; chemical burns, electrical burns or thermal burns; Bartholin's cysts, sinus or abscess; pruritus ani/perianal folliculitis; decubitus ulcers such as bed sores or pressure sores; perianal/sacral ulcers including HIV ulcers, radiation ulcers, ischemic ulcers, and/or malnutrition ulcers; animal or human bites, skin abrasions, lacerations, cellulitis, superficial thrombophlebitis, and loss of skin integrity.

4. The method of claim 1, the metronidazole in a therapeutically effective concentration is about 10 wt %.

5. The method of claim 1, wherein the non-aqueous carrier comprises at least one hydrocarbon compound.

6. The method of claim 5, wherein the hydrocarbon compound comprises white petrolatum (USP).

7. The method of claim 1, wherein said topical composition is in the form of an ointment, lotion, gel, foam or cream.

8. The method of claim 1, wherein metronidazole is applied at a dosage for each application from about 125 mg to about 1250 mg.

9. The method of claim 9, wherein the dosage of metronidazole for each application is from about 125 mg to about 375 mg.

10. The method of claim 1, wherein said topical composition is applied between from 2 to 4 times daily.

11. The method of claim 1, wherein the topical composition has a fluffy texture.

12. The method of claim 1, wherein the metronidazole is the sole active agent.

13. The method of claim 1, further comprising an additional therapeutic agent.

14. The method of claim 13, wherein the additional therapeutic agent is selected from a group consisting of calcium channel blockers, e.g. nifedipine and diltiazem; steroids, e.g. hydrocortisone or a pharmacologically acceptable derivative thereof; analgesic agents, preferably from the amide or ester class such as pramoxine or benzocaine; antimicrobial agents (antibacterial or antiviral), e.g. ciprocfloxacin, amoxicillin-clavulonic acid, erythromycin, tetracycline, clindamycin or doxycycline; substances that either promote skin integrity or inhibits skin breakdown, e.g. vitamin E, aloe, zinc oxide or other barrier cream; anti-inflammatory agents, e.g. a non-steroidal anti-inflammatory agent selected from aminosalicylic acid, ibuprofen, sulindac, piroxicam or diflunisal.

15. The method of claim 1, wherein the topical composition is applied by hand rubbing the composition over the entire area, until the composition disappears.

16. The method of claim 1, wherein the topical composition is applied by physical means including applicator pads, swabs, roller bottles, felt tip or sponge tip applicators.

17. A composition of metronidazole or a pharmacologically acceptable salt thereof at a concentration of about 5 wt % to about 25 wt % in a pharmacologically acceptable carrier for the treatment of tissue damage, wherein the tissue damage is a result of pilonidal cyst and sinus; anorectal Crohns disease including ulcers, fissures, fistulas, skin tags, etc.; post anorectal surgery including hemorrhoidectomy, fistulotomy or incision drainage abscess; thrombosed external hemorrhoid; perineal incision after abdominoperineal resection; pyoderma gangrenosum; erythema nodosum; hidradenitis; non healing surgical incisions; chemical burns, electrical burns or thermal burns; Bartholin's cysts, sinus or abscess; pruritus ani/perianal folliculitis; decubitus ulcers such as bed sores or pressure sores; perianal/sacral ulcers including HIV ulcers, radiation ulcers, ischemic ulcers, and/or malnutrition ulcers; animal or human bites, skin abrasions, lacerations, cellulitis, superficial thrombophlebitis, and loss of skin integrity.
